SEQUENCE LISTING (9 sequences)

SEQ ID NO: 1 - DNA, 36 bp, Artificial Sequence
  misc_binding: oligonucleotides Bae.F for cloning a BaeI recognition site within the HI-loop-encoding sequence
acaactcggtggcggtaccggtgtatacggcggtcc

SEQ ID NO: 2 - DNA, 36 bp, Artificial Sequence
  misc_binding: oligonucleotides Bae.R for cloning a BaeI recognition site within the HI-loop-encoding sequence
ggaccgccgtatacaccggtaccgccaccgagttgt

SEQ ID NO: 3 - DNA, 40 bp, Artificial Sequence
  misc_binding: oligonucleotides LL-Bae-1F for cloning a BaeI recognition site within pKanLL-BaeI
gatcccggtggcggtaccggtgtatacggcggttaataaa

SEQ ID NO: 4 - DNA, 40 bp, Artificial Sequence
  misc_binding: oligonucleotides LL-Bae-1R for cloning a BaeI recognition site within pKanLL-BaeI
gatctttattaaccgccgtatacaccggtaccgccaccgg

SEQ ID NO: 5 - DNA, 12 bp, Artificial Sequence
  misc_binding: MfeI-XbaI-linker
ctagccaattgg

SEQ ID NO: 6 - DNA, 90 bp, Artificial Sequence
  misc_binding: T1, nucleotide sequence encoding the C-domain of Staphylococcus aureus protein A
gcggataacaaattcaacaaagaacaacaaaatgctttctatgaaatcttacatttacctaacttaaacgaagaacaacgtaacggcttc

SEQ ID NO: 7 - DNA, 89 bp, Artificial Sequence
  misc_binding: B1, nucleotide sequence encoding the C-domain of Staphylococcus aureus protein A
gttacgttgttcttcgtttaagttaggtaaatgtaagatttcatagaaagcattttgttgttctttgttgaatttgttatccgcggatc

SEQ ID NO: 8 - DNA, 89 bp, Artificial Sequence
  misc_binding: T2, nucleotide sequence encoding the C-domain of Staphylococcus aureus protein A
atccaaagccttaaagacgatccttcagtgagcaaagaaattttagcagaagctaaaaagctaaacgatgctcaagcaccaaaataata

SEQ ID NO: 9 - DNA, 90 bp, Artificial Sequence
  misc_binding: B2, nucleotide sequence encoding the C-domain of Staphylococcus aureus protein A
ttttggtgcttgagcatcgtttagctttttagcttctgctaaaatttctttgctcactgaaggatcgtctttaaggctttggatgaagcc

(19) United States
(12) Patent Application Publication
(10) Pub. No.: US 2004/0147025 A1
(43) Pub. Date: Jul. 29, 2004

(54) Targeted adenoviral vector displaying immunoglobulin-binding domain and uses thereof

(76) Inventors: Nikolay Korokhov, Birmingham, AL (US); Galina Mikheeva, Houston, TX (US)

Correspondence Address: Thomas J. Kowalski, Esq., c/o FROMMER LAWRENC & HAUG LLP, 745 Fifth Avenue, New York, NY, 10151, US

(21) Appl. No.: 10/624,317

(22) Filed: Jul. 22, 2003

Related U.S. Application Data
(60) Provisional application No. 60/398,057, filed on Jul. 22, 2002.

(51) Int. Cl. A61K 48/00; C12N 15/861; C12N 7/00

(52) U.S. Cl. 435/456; 424/93.2; 435/235.1

(57) ABSTRACT

The present invention provides a targeted recombinant adenovirus vector expressing a fiber protein modified by insertion of an immunoglobulin-binding domain that can crosslink to a fusion protein comprising a targeting ligand and an immunoglobulin Fc domain. Interaction between the immunoglobulin-binding domain and the Fc domain results in a targeted vector::ligand complex, thereby targeting the adenovirus vector to a cell that expresses a cell surface molecule that binds to said targeting ligand.

### CROSS-REFERENCE TO RELATED APPLICATIONS

This non-provisional patent application claims benefit of provisional patent application U.S. Serial No. 60/398,057, filed Jul. 22, 2002, now abandoned.

### BACKGROUND OF THE INVENTION

1. Field of the Invention

The present invention relates generally to the targeting of adenoviral vectors. More specifically, the present invention discloses a targeting strategy that involves genetic modifications of the adenoviral capsid and a protein bridge comprising the Fc-binding domain of Staphylococcus aureus Protein A.

2. Description of the Related Art

Adenoviruses (Ad) are a family of over 50 viral pathogens whose non-enveloped protein capsids embody a single copy of double-stranded DNA genome. Based on their ability to agglutinate red blood cells and the homology of their genomes, adenoviruses have been classified into species A through F. The vast majority of the studies of Ad biology have been done on human Ad of serotypes 2 and 5 (Ad2 and Ad5 respectively), both belonging to species C.

The well studied life cycle of adenoviruses, combined with relatively simple methods for the generation, propagation and purification of recombinants derived from Ad2 and Ad5, has made them attractive candidates as gene delivery vectors for human gene therapy. However, two decades of extensive use of Ad-based vectors as prototypes of future gene therapeutics has revealed a number of limitations that have hampered their rapid transition into the clinic. One of these drawbacks is the relative inefficiency of gene delivery by Ad vectors to certain types of diseased human tissues. On the other hand, the susceptibility of many normal tissues to Ad infection makes them random targets for Ad vectors and results in suboptimal distribution of the viruses upon administration to patients.

Attempts to rectify this deficiency of Ad vectors have been rationalized by the identification of the molecular determinants of virus tropism. A typical Ad capsid is an icosahedron, whose planes are formed by the Ad hexon protein while the vertices are occupied by a penton assembly formed by the penton base and protruding fiber proteins. The cell entry mechanism employed by the majority of human Ad serotypes involves two sequential interactions between an Ad particle and a cell. The first of the two contacts involves the Ad fiber protein and the so-called coxsackievirus-adenovirus receptor (CAR). Specifically, the carboxy terminal knob domain of the fiber binds to the immunoglobulin-like D1 domain of CAR, resulting in tight association of the virus with the cell. The presence of CAR on a target cell is thus recognized as a critical prerequisite of efficient infection. This binding step is followed by the secondary contact involving the arginine-glycine-aspartic acid (RGD) sequence found in the Ad penton base protein with cellular integrins avb3 and avb5. This interaction triggers the internalization of the virion within a clathrin-coated endosome. Acidification of the endosome is believed to lead to the release of the virus into the cytoplasm, followed by its translocation to the nucleus where the replication of the virus begins. It has been reported that while CAR is used by the majority of human Ad as a primary receptor, other cell surface molecules are also exploited in this capacity by certain Ad serotypes. This observation suggests that receptor specificity of a given Ad serotype may be modified by redirecting the virus to alternative cellular receptors.

This targeting concept has been realized by employing the following strategies. In adapter-mediated targeting, the tropism of the virus is modified by an extraneous targeting moiety, the ligand, which associates with the Ad virion either covalently or non-covalently. Adapters or adapter-ligand complexes successfully used for Ad targeting include bispecific antibody (Ab) conjugates, genetic fusions of single chain Ab (scFv) with CAR, or scFv-scFv diabodies (reviewed in Krasnykh and Douglas, 2002). Adapter-mediated targeting is rather versatile and technically simple, it may employ a wide range of targeting ligands, and allows for rapid generation of analytical amounts of targeted complexes and their fast validation. However, it requires the production and purification of at least two different components, the virus and targeting ligand, their subsequent conjugation in a targeting complex, and its purification from non-reacted components. These requirements substantially complicate large-scale production of the vector complex, which may result in significant batch-to-batch variations and complicate the regulatory approval of the vector for clinical use.

In contrast, genetic targeting which is based on genetic incorporation of the ligand into the Ad capsid (reviewed in Krasnykh et al., 2000) results in a one-component, self-assembling and self-replicating vector that may be amplified to any desired scale once it is made and validated. The choice of ligands in this strategy, however, is limited to proteins only. Furthermore, additional limitations may be imposed by the potential structural or biosynthetic incompatibility of the ligand with the protein components of Ad capsid. For instance, recent studies showed that certain protein ligands, such as the epidermal growth factor (EGF) or scFvs whose correct folding requires the formation of disulfide bonds, cannot be used for genetic targeting of Ad.

The prior art is deficient in providing a targeting strategy that would overcome the limitations of the above mentioned targeting methods. The present invention fulfills this long-standing need and desire in the art by developing a new approach that combines elements of genetic modification of the Ad capsid with the adaptor-mediated targeting. Ultimately, this new strategy is expected to result in the development of a one-component vector system consists of an Ad vector expressing a secretory form of a targeting ligand that is secreted into the culture medium during Ad vector propagation and is capable of associating with the progeny virions upon cell lysis. This association is possible due to genetic modifications to both the Ad capsid and the ligand, resulting in a mechanism of self-assembly of the vector:ligand targeting complex.

### SUMMARY OF THE INVENTION

A potential barrier to the development of genetically targeted adenovirus (Ad) vectors for cell. specific delivery of gene therapeutics lies in the fact that several types of targeting protein ligands require posttranslational modifications, such as the formation of disulfide bonds, which are not available to Ad capsid proteins due to their nuclear localization during assembly of the virion. To overcome this problem the present invention develops a new targeting strategy, which combines genetic modifications of the Ad capsid with a protein bridge approach, resulting in a vector::ligand targeting complex. The components of the complex associate by virtue of genetic modifications to both the Ad capsid and the targeting ligand. One component of this mechanism of association, the Fc-binding domain of Staphylococcus aureus Protein A, is genetically incorporated into the Ad fiber protein. The ligand comprises a targeting component fused with the Fc domain of immunoglobulin that serves as a docking moiety to bind to the genetically modified fibers to form the Ad::ligand complex. The modular design of the ligand, and the fact that it is processed via a secretory pathway, solve the problem of structural and biosynthetic compatibility with the Ad, and thus facilitate targeting the vector to a variety of cellular receptors.

The present study shows that targeting ligands incorporating Fc domain and either an anti-CD40 single chain antibody or CD40L form stable complexes with Protein A modified Ad vectors, resulting in significant augmentation of gene delivery to CD40-positive target cells. As this gene transfer is independent of the expression of native Ad5 receptor by the target cells, this strategy results in the derivation of truly targeted Ad vectors suitable for tissue-specific gene therapy.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1: Analysis of the transiently expressed fiber-Cd (C domain) proteins. 293T/17 cells transfected with pVS-derived expression plasmids were lysed and aliquots of the lysates containing 5mg of total soluble protein were loaded on an SDS-PAGE gel in sample buffer. The fiber proteins in some of the samples were fully denatured by heating for 5 min at 96° C. (lanes b). These samples were expected to contain the fiber monomers only. In parallel, similarly prepared samples analyzed under “semi-native” conditions were not heat-denatured (lanes a) and were supposed to contain the fiber-Cd proteins in a trimeric configuration. Upon separation, the proteins were electroblotted onto PVDF membrane and probed with anti-fiber tail mAb 4D2.

FIG. 2: Assessment of the Fc- and CAR-binding ability of the transiently expressed fiber-Cd proteins. The bait proteins, Fc-G28.5 (FIG. 2A) or recombinant CAR (FIG. 2B), adsorbed on ELISA plates were probed with serial dilutions of lysates of fiber-Cd expressing 293T/17 cells. The quantity of the recombinant fibers used in the assay were normalized according to the concentration of total soluble protein in the lysates. The bait-bound fibers were then detected with anti-fiber mAb followed by HRP-conjugated anti-mouse immunoglobulin G antibodies.

FIG. 3: Characterization of Ad virions incorporating fiber-Cd proteins. FIG. 3A shows Western blotting of Cd-modified Ad. Aliquots equal to 10¹⁰ vp of CsCl-purified Ad vectors were boiled in the sample buffer and their protein components were separated on an SDS-PAGE gel. The fibers electrotransferred onto a membrane were identified with anti-fiber tail mAb 4D2. Lane 1, Ad5.DR-HI-Cd; lane 2, Ad5.DR-HI10-Cd; lane 3, Ad5.DR-HI40-Cd; lane 4, Ad5.DR-HI80-Cd; lane 5, Ad5.DR-LL-Cd; lane 6, Ad5.DR. FIG. 3B shows binding of Cd-containing Ad vectors to Fc-modified targeting ligand. The ligand, Fc-G28.5, was adsorbed on an ELISA plate and incubated with aliquots of the purified Cd-modified Ad virions ranging from 1×10⁹ to 3×10¹¹ vp. Fc-bound Ad particles were detected with anti-Ad2 polyclonal antibodies.

FIG. 4: Ligand-mediated transduction of CD40-positive cell targets. 293.CD40 (FIG. 4A) or 293 (FIG. 4B) cells preincubated with either Ad5 fiber knob protein, fiber knob and Fc-G28.5 protein, or plain medium were infected with each of the Cd-modified vectors at an MOI of 10 vp/cell. Ad5DR vector incorporating wild type Ad5 fibers was used as a control. The bars correspond to the luciferase activity in relative light units (RLU) detected in transduced cells 24 hrs post infection (average activity obtained in three replicates). The error bars show standard deviations.

FIG. 5: Incorporation of Fc-G28.5 fusion protein into targeting vector complexes. Targeting complexes formed by association of the Fc-G28.5 ligand with either Ad5.DR-HI10-Cd, Ad5.DR-HI40-Cd, or Ad5.DR-LL-Cd were purified from unincorporated ligands on CsCl gradients and aliquots of each preparation corresponding to 1.5×10⁹ vp were analyzed by immunoblotting alongside samples of Ad vectors which were not incubated with Fc-G28.5. FIG. 5A shows the membrane probed with anti-fiber mAb, FIG. 5B demonstrates the result of the ligand detection done with Penta-His mAb. “+” indicates the samples pre-incubated with the ligand; “−” shows those containing the Ad vectors only; C, a mixture of 1.5×10⁹ vp of Ad5.DR with 12ng of Fc-G28.5.

FIG. 6: Transduction of cells by the preformed targeted vector complexes. CD40-negative 293 (FIG. 6A) or CD40-positive Namalwa (FIG. 6B) cells were infected with either Ad5.DR-HI40-Cd, or Ad5.DR-LL-Cd at an MOI of 10 vp/cell or 500 vp/cell respectively. Each of the Cd-modified vectors was used either alone (indicated by “−”) or in association with the Fc-G28-5 ligand (shown by “+”). Ad5.DR was used as an unmodified vector control. The infection was done with or without recombinant Ad fiber knob protein being added to the incubation mixture. Luciferase activity in the transduced cells is shown as either the percentage of the activity detected in unblocked samples (FIG. 6A), or in RLU (FIG. 6B). Standard deviations are represented by the error bars. Of note, the absolute values of luciferase activity in 293 cells infected with targeted vectors were significantly lower than those seen upon infection with untargeted viruses.

FIG. 7: Ligand-mediated inhibition of gene transfer by Ad5.DR-LL-Cd::Fc-G28.5 vector complex. CD40-positive Namalwa cells pre-incubated with medium alone or with increasing concentrations of the Fc-G28.5 ligand were transduced with the preformed Ad5.DR-LL-Cd::Fc-G28.5 vector at an MOI of 100 vp/cell. Ad5.DR vector containing unmodified fiber was used as a negative control. Luciferase activity detected in the lysates of cells transduced with the viruses in the presence of competing ligand protein was normalized to that in the cells infected in the absence of free Fc-G28.5. The data points represent the results of three independent determinations with the error bars corresponding to standard deviations.

FIG. 8: Targeted transduction of human monocyte-derived dendritic cells. Dendritic cells derived from human monocytes were transduced with either Ad5.DR (shown as Fb wt) or Cd-modified Ad5.DR-LL-Cd vector. In the latter instance, the vector was used in either the untargeted form or pre-complexed with one of the targeting ligands, Fc-G28.5 or Fc-CD40L. Recombinant Ad5 fiber knob or/and Fc-G28.5 proteins were added to some samples to block the interaction between the virus and the CAR or CD40, respectively. Each data point is an average of two measurements. The error bars show standard deviations.

FIG. 9 shows overall design of CD40-targeted Ad vector. FIG. 9A shows Ad virion and fiber:ligand complex. Each of the three polypeptides constituting the fiber trimer contains a protein tag (C-domain of S. aureus protein A) incorporated within its knob domain. Similarly, each ligand molecule (TNF-like domain of CD40L or anti-CD40 scFv) contains a complementary tag (Fc-domain). Interaction between the two complementary tags results in cross-linking the virus with the ligand. Only one fiber polypeptide is shown as tag-modified. FIG. 9B shows the genome of PSMA-expressing, CD40-targeted Ad vector. The E1 and E3 regions of the Ad genome are replaced with a double expression cassette containing prostate-specific membrane antigen (PSMA)- and ligand-encoding genes, and the green fluorescent protein (GFP) gene, respectively. The wild type fiber gene is modified to express a tagged form of the fiber protein.

### DETAILED DESCRIPTION OF THE INVENTION

The present invention describes an adenoviral vector targeting approach that combines the advantages of the previously established protein bridge-mediated and genetic modification of virus tropism. It is an object of the present invention to develop an Ad vector system in which genetic modifications done to both the Ad vector capsid and secretory ligand would allow them to self-associate into a stable complex.

This approach was dictated by the major limitation to genetic targeting of Ad, which otherwise remains the most straightforward and efficient way to modify Ad tropism. This limitation is the structural and biosynthetic incompatibility of the protein components of Ad capsid, including the receptor-binding fiber, with certain types of protein molecules that could be attractive candidates as Ad targeting ligands. These candidate proteins include a number of naturally existing molecules (both secretory and anchored within the cell membrane) that require extensive posttranslational modifications that are not available to the Ad proteins localized within the nucleus of infected cells. The major structural feature which limits the use of these proteins as Ad ligands is the presence of the disulfide bonds in their molecules. These disulfide bonds can only be formed in the oxidative environment of the endoplasmic reticulum (ER) by disulfide isomerases, which are residents of the ER. Soon after translation, the fiber and other proteins constituting the Ad capsid traffic to the nucleus whose reducing environment prevents the formation of disulfide bonds. Obviously, the same would hold true for any extraneous protein genetically fused with the fiber. Redirecting the fiber to endoplasmic reticulum, although technically feasible, does not solve the problem as the fiber is then excluded from the assembly of the progeny Ad virions that takes place in the nucleus. These considerations and limitations were proven lately in a report that showed two types of ligands containing disulfide bonds, the epidermal growth factor and scFv, cannot be genetically fused with the functional fiber.

This incompatibility of desired targeting ligands with Ad proteins is resolved in the present work by allowing the virus and the ligand to follow their natural biosynthetic pathways in a non-conflictual manner and, upon proper folding and assembly, associate in a functional vector complex. Data presented herein establish the feasibility of this concept by showing that individual components of such a binary system may be engineered and then put together to form a targeted vector. In one embodiment, the molecular constituents for self-assembly used in the present study are the Fc domain of human immunoglobulin and the Fc-binding domain of Staphylococcus aureus Protein A, which are used to modify the ligand and, the virus respectively. The natural affinity of the Protein A for Fc underpins the targeted complex formation. The 59 amino acids long domain C of Protein A was incorporated into either the HI loop or the carboxy terminus of Ad5 fiber to create a docking site for a Fc-modified targeting ligand. None of the modifications affected the yield or the growth dynamics of the resultant Ad vectors. The engineered fibers could be incorporated into mature Ad virions very efficiently. Apparently, none of these modifications caused any significant changes in the folding of the fiber; as its binding to natural Ad receptor, CAR, which requires the involvement of amino acid residues localized on two knob subunits, was not affected.

The Fc domain of Ig fused with the ligand served a double duty: in addition to being a facilitator for the expression and secretion of the ligand, it also functioned as an element of the two-component mechanism mediating the association of the ligand with the virus. The Fc domain of Ig has long been used for the purposes of recombinant protein expression. Its incorporation into a protein of interest normally results in a substantial increase in the yield of the protein and also greatly simplifies the purification of the fusion protein on Protein A-containing matrixes. Thus, the use of Fc domain in the present study allowed one to produce secretory form of the targeting ligand in substantial amounts and easily purify it by affinity chromatography. When mixed together, the virus and the ligand undergo self-assembly into a targeting complex that can be purified from unincorporated ligand and then stored as a ready-to-use reagent while retaining its gene delivery properties.

As shown in results from an in vitro gene transfer assay, the pre-formed complexes of Ad with Fc-tagged anti-CD40 scFv or CD40L showed selective gene transfer to target cells via the CD40-mediated pathway. Importantly, the present invention demonstrates that association with the targeting ligand results in structural interference with the CAR binding site within the knob, thereby rendering the vector complexes truly targeted. Subsequent use of these CD40-targeted vectors to infect human monocyte-derived dendritic cells demonstrated an augmentation of overall gene transfer that was 30-fold higher than that achieved with an isogenic control Ad incorporating unmodified, wild type fibers, suggesting that the vectors designed in this study may be a more efficient means of delivering antigen-encoding genes to dendritic cells for genetic immunization.

The present invention is a new version of the protein bridge-based targeting approach that offers significant advantages over previously described methods. For instance, by providing a universal solution for the expression of secretory targeting ligands, the targeting approach disclosed herein favorably compares to previously used strategy employing chemical cross-linking of antibodies to form targeting conjugate. Generation of those chemical cross-linked conjugates was proved to be inefficient and thus required large amounts of starting components. Reproducibility in the yields of the cross-linked conjugates is also an issue. The high degree of structural similarity of Ad fiber knob domains from different serotypes predicts the compatibility of Protein A domain C with the frameworks of fiber knobs other than that of Ad5.

The most significant advantage of the strategy described herein is that it allows for the generation of targeted Ad vector in a single infection procedure, wherein the Ad vector modified with the Protein A domain C also expresses the targeting ligand comprising a Fc portion. Targeting complexes self-formed upon cell lysis by the virus progeny will then be isolated by the protocols established for Ad purification. This would significantly simplify the vector manufacturing process and result in high reproducibility and low production costs. The fact that both the virus and the ligand can be produced using the same method, i.e. infection of 293 cells with Ad, strongly supports the feasibility of the proposed approach. While the C domain-modified Ad vectors described herein were designed to be targeted with Fc-ligand fusion proteins, the present invention would be fully suitable for vector targeting utilizing full size antibodies as well.

Targeting Adenoviral Vectors for Genetic Anti-Cancer Immunization

The present invention would be useful in the development of genetic anti-cancer immunization. The development of anti-cancer vaccination strategies has been rationalized by the recent identification of tumor associated antigens (TAA) which may be recognized by the immune system as specific markers of cancer cells, thereby identifying these cells as the targets. These tumor associated antigens include proteins encoded by genes with mutations or rearrangements unique to tumor cells, reactivated embryonic genes, tissue-specific differentiation antigens, and a number of other self proteins. However, despite the identification of these targets, development of effective anti-cancer vaccination strategies has been limited to a large extent by the lack of means for successful vaccination against these weak, self-derived antigens. The generation of a potent anti-tumor associated antigen immune response is thus recognized as a key issue in the development of efficient anti-cancer immunization strategies.

The problem of poor immunogenicity of self-derived tumor-associated antigens can be overcome by efficient antigen presentation by dendritic cells. Current understanding of the mechanisms of immune response development suggests that efficient capture and presentation of tumor associated antigens by antigen presenting cells (APCs) is a pivotal step in eliciting strong anti-cancer immunity. In this regard, dendritic cells (DCs), so-called “professional” APCs, play a major role in the induction of an immune response due to their ability to process and present antigen, express high levels of co-stimulatory molecules, and activate both CD4⁺ and CD8⁺ naive T lymphocytes.

Dendritic cells represent a heterogeneous population of bone marrow-derived cells present at low numbers in most peripheral tissues, where they continuously sample the antigenic content of their environment by phagocytosis, macropinocytosis and receptor-mediated endocytosis. A captured antigen is then processed intracellularly, being degraded into short peptides that are loaded onto class I and class II major histocompatibility (MHC) molecules for subsequent display on the cell surface. When dendritic cells encounter local inflammatory mediators, such as tumor necrosis factor a (TNFa) or bacterial lipopolysaccharide, they become activated and undergo a series of physiologic changes leading to their terminal differentiation, a process called “dendritic cell maturation”.

Dendritic cell maturation includes redistribution of MHC molecules from intracellular endocytic compartments to the cell surface, a selective decrease of antigen and pathogen internalization activity and a marked increase in surface expression of co-stimulatory molecules for T cell activation. Maturation also entails profound changes in dendritic cell morphology, reorganization of their cytoskeleton and surface expression of several integrins and chemokine receptors that determine their migration from peripheral tissues to secondary lymphoid organs. Thus, dendritic cells serve as initiators of immune response, capturing antigen at portals of entry and delivering it in a highly immunogenic form for efficient display to T cells.

Stemming from their key functions as central mediators of T cell-based immunity, the uses of dendritic cells have been proposed in a number of clinical immunotherapy strategies. In order to increase the efficiency of delivery of tumor associated antigen-encoding genes to dendritic cells, natural mechanisms of virus-mediated transduction of dendritic cells have been employed. To this end, recombinant adenoviral (Ad) vectors have proved to be more efficient in delivering tumor associated antigen-encoding sequences into dendritic cells than traditional transfection methods.

Several years of studies employing Ad vectors for transduction of dendritic cells, however, have resulted in rather controversial data on the efficiency of this method. A critical analysis of the literature reveals that in those instances where significant levels of Ad-mediated gene transfer to dendritic cells was reported, very high multiplicities of infection (MOIs) had to be used. For instance, Dietz et al. (Blood 91:392, 1998) reported high efficiency adenovirus-mediated gene transfer to human dendritic cells using Ad vector at a MOI of 5,000 virions per cell. Similarly, in order to achieve efficient transduction of bone marrow-derived murine dendritic cells with Ad, Kaplan et al. (J. Immunol. 163:699, 1999) used an MOI of 500 infection units per cell, and Rea et al. transduced human dendritic cells at a MOI of 1,000 plaque forming units per cell (J. Virol. 73:10245, 1999). Whereas the need to use such high doses of the vector does not normally constitute a problem in “proof of concept” studies done in a laboratory, it prevents broad application of Ad-transduced dendritic cells as therapeutic vaccines in the clinic. Importantly, the exposure of immature dendritic cells, whose primary biological function is to capture antigen, to a high concentration of Ad vectors may result in the capture of Ad virions by the dendritic cells and elicitation of an anti-Ad rather than the desired anti-TAA immune response expected from the transduction. While these considerations may not present problems with respect to ex vivo immunization of dendritic cells with Ad vectors, they are particularly important in the context of potential application of Ad-mediated transduction of dendritic cells in vivo, where high doses of Ad vectors administered to patients may cause severe side effects due to toxicity (25-29), thereby compromising the efficiency of the treatment. Thus, any significant improvement on Ad vectors' capacity to transduce dendritic cells that would allow utilization of lower viral doses with higher rates of gene transfer would be highly beneficial for the field of genetic immunization.

Recent studies designed to address the resistance of dendritic cells to Ad infection have revealed the molecular basis of this problem. A majority of human Ad utilizes a cell entry pathway that involves the primary cellular receptor, the coxsackievirus-adenovirus receptor (CAR). Expression of CAR below certain threshold levels may be a common reason for the Ad-refractoriness of a variety of cell targets. Specifically, poor efficiencies of gene transfer to dendritic cells by Ad vectors have been shown to correlate with low levels of CAR expression in these cells. Therefore, the dependence of Ad-mediated transduction on the levels of CAR expressed on target dendritic cells represents a major obstacle in using Ad vectors for genetic immunization.

CAR-deficiency of dendritic cells and their refractoriness to Ad infection may be overcome by modification of Ad tropism to target the vector to specific receptors expressed by dendritic cells. Recent studies performed at the Gene Therapy Center at University of Alabama at Birmingham have clearly demonstrated the efficacy of this tropism modification strategy by targeting the vector to the CD40 receptor present on the surface of dendritic cells. Specifically, by employing a bispecific antibody with affinities for both the adenovirus fiber knob and CD40, a luciferase-expressing Ad vector was re-routed via CD40 that served the role of an alternative primary receptor for Ad binding. The selection of CD40 as an alternative receptor for the Ad vector was rationalized by the fact that this molecule, which play an important role in antigen-presentation by dendritic cells, is efficiently expressed by immature dendritic cells. The CD40-targeted Ad vector increased reporter gene expression in dendritic cells by at least two orders of magnitude as compared to untargeted Ad. Furthermore, this enhancement was blocked by ˜90% when cells were pretreated with an excess of the unconjugated anti-CD40 monoclonal antibody.

Importantly, this antibody-based targeting resulted in modulation of the immunological status of dendritic cells by inducing their maturation. This was demonstrated phenotypically by increased expression of CD83, MHC, and costimulatory molecules, as well as functionally by production of IL-12 and an enhanced allostimulatory capacity in a mixed lymphocyte reaction (MLR). It has been reported that activation of dendritic cells to maturity renders them resistant to the effects of dendritic cell inhibitory cytokines like IL-10 as well as to direct tumor-induced apoptosis. The capacity with which murine dendritic cells can generate an immune response in vivo has been shown to correlate with the degree of their maturation. Moreover, based on proposals that CD40 activation may bypass CD4⁺ T cell help, a CD40-targeted Ad might also have applications in cases of CD4⁺ dysfunction. The dual role of CD40 in this schema as both a surrogate Ad receptor and a powerful trigger of DC maturation rationalize further development of dendritic cell-targeting Ad vectors for anti-cancer immunization.

Alternatively, an Ad vector may be targeted to CD40 by cross-linking with the natural ligand for CD40 receptor, CD40 Ligand or CD40L. CD40-CD40L interaction is characterized by high affinity and specificity and also launches a cascade of events leading to the initiation of an immune response. The multivalent interaction of trimeric CD40L with CD40 receptors causes CD40 ligation, which then results in enhanced survival of these cells and secretion of cytokines such as IL-1, IL-6, IL-8, IL-10, IL-12, TNF-a, MIP-1a and enzymes such as matrix metalloproteinase. CD40-CD40L interaction also enhances monocyte tumoricidal activity. In addition, ligation of CD40 to CD40L considerably alters dendritic cell phenotype by upregulating the expression of costimulatory molecules such as CD54/ICAM-1, CD58/LFA-3, CD80/B7-1, and CD86/B7-2. Therefore, the interaction between CD40 and CD40L has important consequences for both antigen presenting cell function and T cell function.

The present invention discloses an Ad vector suitable for selective and efficient gene transfer to dendritic cells. The targeting system involves interaction between the Fc domain of an antibody and an immunoglobulin-binding domain to cross-link an adenoviral vector to a targeting ligand. The Ad vector is targeted to CD40, which functions as a surrogate viral receptor, by complexing the Ad vector with a CD40-specific protein moiety such as the natural ligand for CD40, CD40L, or an anti-CD40 single chain antibody. A single-chain (scFv) version of anti-human CD40 mAb G28.5 has been derived at the Gene Therapy Center at University of Alabama and its ability to bind CD40 expressed on cell surface has been demonstrated. As this scFv represents the CD40-binding domains of the parental mAb, by all accounts it should retain the capacity of G28.5 to activate dendritic cells upon binding to CD40 and may thus be used as an adequate substitute for the full size mAb in a targeting strategy. Fc domain of an antibody and the C domain of S. aureus protein A (CdpA) are incorporated into the targeting ligand and the Ad fiber protein respectively, and interaction between these two complementary tags results in cross-linking the virus with the targeting ligand. To date, the carboxy terminus and the HI loop within the Ad fiber knob domain have been identified as favoring incorporation of heterologous peptide sequences. Recent work has demonstrated that each of these sites within the fiber can accommodate polypeptide sequences exceeding 70 amino acid residues in length.

In addition to the C domain of S. aureus protein A, one of skills in the art can use other immunoglobulin-binding domains well known in the art.

In addition to attaching an immunoglobulin-binding domain to the fiber protein, the immunoglobulin-binding domain can also be inserted into fiber-fibritin chimera as an alternative strategy. The fiber-fibritin protein was designed so that the structure of the domain providing for trimerization of the chimera (fibritin) is not affected by incorporation of heterologous peptides/polypeptides within the protein, thereby dramatically increasing the odds of obtaining stable derivatives of this “backbone” molecule.

Targeted Adenoviral Vectors Expressing Prostate Cancer-Specific Tumor Antigen

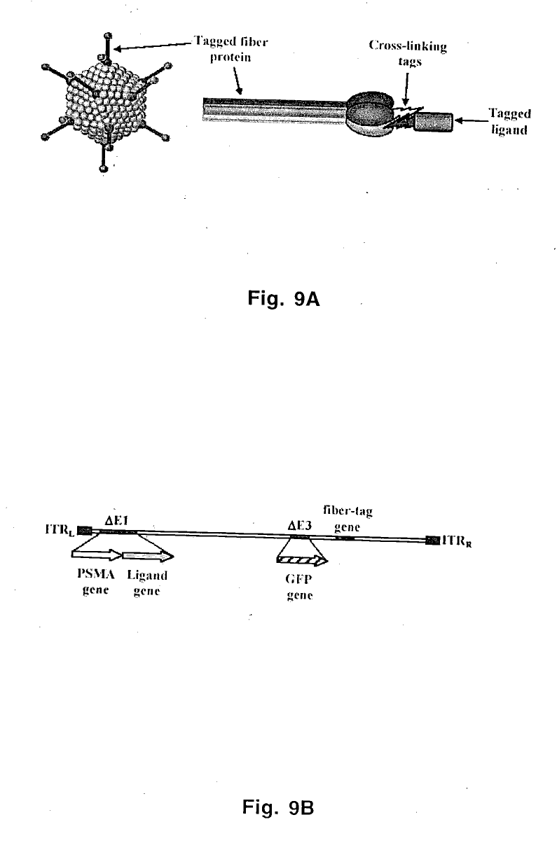

One object of the present invention is to provide targeted adenoviral vectors for uses in immunotherapy. Accordingly, in one embodiment of the present invention, there is provided a highly efficient Ad vectors suited for genetic immunization of humans against prostate cancer (PCA) (FIG. 9). The rationale of this approach is based on the fact that a potent anti-prostate cancer immune response can be induced by selective and efficient delivery to, and expression in, human dendritic cells of a prostate cancer-specific antigen, prostate-specific membrane antigen (PSMA). It is expected that efficient expression of PSMA within dendritic cells, which are highly specialized, professional antigen-presenting cells, would lead to induction of anti-PSMA immune response directed against prostate cancer tumor and eradication of tumor cells by the patient's immune system.

This expectation is based on the following findings. Prostate tumors express tumor-specific antigens (TAAs) that are suitable for development of immune-based therapies (Tjoa and Murphy, 2000). Cytotoxic lymphocytes (CTLs) have been generated in vitro against prostate-specific antigen (PSA). Importantly, more recent data demonstrate that PSA-specific cellular immunity can be generated in humans (Meidenbauer et al., 2000). Immunotherapy has been successfully employed to treat prostate tumors in mouse models. Dendritic cells have been shown to be effective in generating prostate tumor-specific immunity in humans in other contexts as well (Salgaller et al., 1998). A recent report suggested that dendritic cells pulsed with mRNA from prostate carcinomas induced significant human immunity that correlated with reduced metastatic tumor transit in blood (Heiser et al., 2002).

PSMA is a prostate cancer tumor-specific antigen, which is produced by both the prostate cancer tumor cells and the endothelial cells of the prostate cancer tumor vasculature, that is the subject of immune attack by CTLs (Lodge et al., 1999). Dendritic cells pulsed with PSMA-specific peptides have generated significant short-term clinical responses in human patients (Murphy et al., 1999), prompting further employment of this tumor-specific antigen in development of immunotherapies for prostate cancer patients (Tasch et al., 2001). Interestingly, antibodies directed against PSMA are also effective in treating prostate cancers, with anti-PSMA immunity being associated with tumor clearance in mice. Both cellular and humoral immunity may be important, and dendritic cells are capable of inducing both types of responses. Expression of PSMA by both the prostate tumor cells and prostate vasculature endothelium suggests. that genetically induced anti-PSMA immunity will cause the destruction of the tumor directly and also via abrogation of its blood supply, thereby resulting in a synergistic enhancement of the therapeutic effect. Thus, based on these data, strategies to target PSMA expression to dendritic cells may improve the effectiveness of immune-based therapies for cancer of prostate.

The major improvement of the Ad vector disclosed herein compared to the Ad5-based vectors presently used for anti-prostate cancer vaccination is its engineered ability to deliver PSMA to human dendritic cells in a targeted, highly efficient manner. Based on early findings by Tillman et al. (1999, 2000), not only it is expected to dramatically increase the efficiency of dendritic cells transduction by the CD40-targeted Ad, it is also expected that binding of this Ad to CD40 on dendritic cells will trigger their maturation and the ability to activate cytotoxic T cells, thereby leading to development of a potent anti-prostate cancer immune response. The vector of the present invention is engineered to express PSMA, and a secretory, tagged form of a targeting ligand. In its final configuration it will consist of a recombinant form of either CD40L or an anti-CD40 scFv linked via Fc:protein A interaction to an Ad virion encoding PSMA. Of note, the Fc domain-containing ligands will be encoded by the genomes of the same Ad vectors they are designed to associate with and thus retarget. Importantly, in the described configuration this vector will constitute a one-piece, self- assembling delivery vehicle, production of which does not require any additional steps over and above its amplification in a corresponding cell line with subsequent purification. This feature of the proposed system should greatly facilitate large-scale manufacturing of the targeted vector by eliminating the need for production of the vector and the targeting ligand in two separate technological processes.

In view of the present disclosure, one of ordinary skill in the art would readily apply the method of the instant invention to direct adenoviral vectors carrying various heterologous proteins or tumor-specific antigens to targets besides CD40⁺ cells. Other targeting ligands and heterologous proteins or TAA that are within the scope of the instant invention would be readily recognized by a person having ordinary skill in this art.

In accordance with the present invention there may be employed conventional molecular biology, microbiology, and recombinant DNA techniques within the skill of the art. Such techniques are explained fully in the literature. See e.g., Maniatis, Fritsch & Sambrook, “Molecular Cloning: A Laboratory Manual (1982); “DNA Cloning: A Practical Approach,” Volumes I and II (D.N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The term antibody used herein is intended to encompass both polyclonal and monoclonal antibodies. The term antibody is also intended to encompass whole antibodies, biologically functional fragments thereof, chimeric and humanized antibodies comprising portions from more than one species.

Biologically functional antibody fragments include Fab, Fv, F(ab′)₂, and scFv (single-chain antigen-binding protein) fragments. As used herein, single chain antibodies or scFvs are polypeptides which consist of the variable (V) region of an antibody heavy chain linked to the V region of an antibody light chain with or without an interconnecting linker. This comprises the entire antigen binding site, and is the minimal antigen binding site.

Chimeric antibodies can comprise proteins derived from two different species. The portions derived from two different species can be joined together chemically by conventional techniques or can be prepared as a single contiguous protein using genetic engineering techniques (See, e.g., Cabilly et al., U.S. Pat. No. 4,816,567, Neuberger et al., WO 86/01533 and Winter, EP 0,239,400). Such engineered antibodies can be, for instance, complementarity determining regions (CDR)-grafted antibodies (Tempest et al., Biotechnology 9:266-271 (1991)) or “hyperchimeric” CDR-grafted antibodies which employ a human-mouse framework sequence chosen by computer modeling (Queen et al., Proc. Natl. Acad. Sci. USA 86:10029-10033 (1989)).

The present invention is directed to a targeted recombinant adenovirus vector comprising (i) a gene encoding a heterologous protein; (ii) a modified fiber protein with an immunoglobulin-binding domain; and (iii) a gene encoding a fusion protein comprising an immunoglobulin Fc domain and a targeting ligand. Binding of the immunoglobulin-binding domain to the Fc domain would connect the targeting ligand to the modified fiber protein, thereby targeting the adenovirus vector to a cell that expresses a cell surface molecule that binds to the targeting ligand. The modified fiber protein can be a fiber-fibritin chimera. The immunoglobulin-binding domain (for example, the Fc-binding domain of Staphylococcus aureus Protein A) can be inserted at the HI loop or the carboxy terminal of the modified fiber protein. In one embodiment of the present invention, the adenovirus vector is targeted to CD40⁺ cells, such as dendritic cells, by employing CD40 ligand or a single chain fragment (scFv) of anti-human CD40 antibody as targeting ligand.

The present invention is also directed to a method of gene transfer to CD40⁺ cells using the CD40-targeted adenoviral vector disclosed herein. In general, the CD40⁺ cells are dendritic cells.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Cell Lines and Reagents

293 human embryonal kidney cells, their derivative 293T/17 which expresses the simian virus 40 large T antigen, and Namalwa Burkitt's lymphoma human cells were purchased from the American Type Culture Collection (Manassas, Va). Namalwa cells were cultured in RPMI medium adjusted to contain 1.5 g/L sodium bicarbonate, supplemented with 2 mM L-glutamine, 4.5 g/L glucose, 1.0 mM sodium pyruvate, and 7.5% fetal bovine serum (FBS). 293 and 293T/17 cells were propagated in Dulbecco's modified Eagle's medium (DMEM)/F-12 medium with 10% FBS, 2 mM glutamine, 100 U/ml penicillin, and 100 mg/ml streptomycin. FBS was purchased from HyClone (Logan, Utah), and media and supplements were from Mediatech (Herndon, Va.). All cells were propagated at 37° C. in a 5% CO₂ atmosphere.

Dendritic cells (DCs) were derived from the peripheral blood of normal donors. Peripheral blood mononuclear cells were purified with gradient centrifugation using Histopaque (Sigma Diagnostics, St. Louis, Mo.). CD14-positive monocytes were then isolated using CD14 microbeads and magnetic cell sorting (Miltenyi Biotec, Auburn, Calif.). They were cultured for six days in RPMI 1640 medium with 10% FBS, 2 mM glutamine, 100 U/ml penicillin, 100 ug/ml streptomycin, and 50 mM 2-ME containing 100 ng/ml recombinant human IL-4 (R&D Systems, Minneapolis, Minn.) and 100 ng/ml recombinant human GM-CSF (Immunex, Seattle, Wash.). Expression of molecular markers typical of immature DC (CD14 negative; CD11c, CD40, CD86, and HLADR positive) was confirmed by staining with relevant monoclonal antibodies (mAb).

Rabbit anti-Ad2 polyclonal antibodies were purchased from the National Institute of Allergy and Infection Diseases (Bethesda, Md.). Anti-mouse and anti-rabbit immunoglobulin polyclonal antibodies conjugated with horseradish peroxidase were from Amersham Pharmacia Biotech Inc. (Piscataway, N.J.) and DAKO (Carpinteria, Calif.), respectively. 4D2 anti-fiber mouse mAb (Hong and Engler, 1996) was provided by Jeffrey Engler (University of Alabama at Birmingham, Ala.). Penta-His mAb, which binds five histidine sequence was purchased from Qiagen (Valencia, Calif.).

Restriction endonucleases and T4 DNA ligase were purchased from New England Biolabs (Beverly, Mass.). The polymerase chain reaction (PCR) was performed with Pfu DNA polymerase (Stratagene, La Jolla, Calif.)

### EXAMPLE 2

Design of Ad5 Fiber Protein Modified with the C Domain of Staphhylococcus aureus Protein A

To design a versatile mechanism for attachment of targeting ligands to Ad particles, the structure of each of these components were modified with distinct protein moieties capable of forming stable heteroduplexes upon association with each other. To this end, the C domain (Cd) of Staphylococcus aureus Protein A was introduced within the fiber protein of the Ad5 vector. This domain is known to bind with high selectivity and affinity to the Fc domain of immunoglobulins (Ig). Therefore, Ad virions incorporating such Cd-modified fibers were expected to bind targeting ligands designed to contain an Fc domain.

A total of five genes coding for different C domain (Cd)-containing fibers were designed by incorporation of the C domain open reading frame into either the carboxy terminus of the fiber protein (Fb-LL-Cd), or into the HI loop of its knob domain. In the latter instance, in addition to direct fusion of the C domain sequence with that of the HI loop (Fb-HI-Cd), three other constructs (Fb-HI10-Cd, Fb-HI40-Cd and Fb-HI80-Cd) were made in which the C domain was flanked within the loop with flexible linkers derived from the AdS penton base protein (Belousova et al., 2002). These additional constructs were designed to avoid potential steric hindrance that could be caused by the proximity of the knob to C domain within the fusion molecule. The C domain was extended away from the knob by linkers having 5, 20 or 40 amino acid residues.

### EXAMPLE 3

Vectors for Ad5 Fiber Protein Modified with the C Domain of Staphylococcus aureus Protein A

To facilitate modifications of the HI-loop of Ad5 fiber, shuttle vector pKanHI-BaeI carrying the Ad5 fiber gene with flanking regions of Ad genomic DNA and the recognition sequence for the restriction endonuclease Bae I within the HI-loop was constructed by a two-step cloning strategy. First, the shuttle vector pKan_{ρ}HI was generated by subcloning of the 3.1-kb PmeI-EcoRI fragment of pXK_{ρ}HI (Belousova et al., 2002), whose ends were filled-in with the Klenow fragment of DNA polymerase I of E.coli, into ApoI-AflIII-digested pZErO-2 (Invitrogen, Carlsbad, Calif.). Next, a BaeI recognition site within the HI-loop-encoding sequence was generated by cloning the duplex made with oligonucleotides BaeI (ACAACTCGGTGGCGGTACCGGTGTATACGGCGGTCC, SEQ ID NO. 1) and Bae.R (GGACCGCCGTATACACCGGTACCGCCACCGAGTTGT, SEQ ID NO. 2) into EcoRV-digested plasmid pKan_{ρ}HI, resulting in shuttle vector pKanHI-BaeI.

A shuttle vector suitable for modifications of the carboxy terminus of the fiber protein was designed by subcloning an AgeI-MfeI-fragment of the previously described pBS.F5_{LL}BamHI (Krasnykh et al., 1996) into the AgeI-MfeI-digested pKan_{ρ}HI. This resulted in plasmid pKanLL-BamHI encoding a modified fiber with a C-terminal peptide linker (G₄S)₃ followed by a BamHI restriction site. This site was then replaced with the BaeI recognition sequence by inserting a duplex made of two oligonucleotides, LL-Bae-1F (GATCCCGGTGGCGGTACCGGTGTATACGGCGGTTAATAAA, SEQ ID NO. 3) and LL-Bae-1R (GATCTTTATTAACCGCCGTATACACCGGTACCGCCACCGG, SEQ ID NO. 4), thereby generating pKanLL-BaeI.

Plasmid pDV67, which was constructed for the expression of Ad5 fiber and its derivatives in mammalian cells, was described in Von Seggern et al. (2000). To simplify the transfer of fiber genes assembled within pDV67 into the pKan3.1-derived fiber shuttle vectors, the MfeI restriction site located upstream from the CMV promoter was deleted to make pVSI. A new MfeI site was introduced downstream from the 3′ end of the fiber open reading frame (ORF) by cloning an MfeI-XbaI-linker (CTAGCCAATTGG, SEQ ID NO. 5) into XbaI-digested pVSI, resulting in pVSII.

Recombinant genes encoding the Ad5 fiber modified by incorporation of the C-domain of Staphylococcus aureus Protein A (SpA) within the HI loop and at the carboxy(C)-terminus were assembled in two steps. First, AgeI-MfeI-fragments isolated from the plasmids pKanHIBae1, pKan-LL-Bae1, pHI.PB10, pHI.PB40, or pHI.PB80 (3), were cloned into AgeI-MfeIdigested pVSII. Next, the nucleotide sequence encoding the C-domain of SpA was assembled with two pairs of oligonucleotides T1 (GCGGATAACAAATTCAACAAAGAACAACAAAATGCTTTCTATGAAATCT TACATTTACCTAACTTAAACGAAGAACAACGTAACGGCTTC, SEQ ID NO. 6), B 1 (GTTACGTTGTTCTTCGTMAAGTTAGGTAAATGTAAGATTTCATAGAAA GCATTTTGTTGTTCTTTGTTGAATTTGTTATCCGCGGATC, SEQ ID NO. 7) and T 2 (ATCCAAAGCCTTAAAGACGATCCTTCAGTGAGCAAAGAAATTTTAGCAG AAGCTAAAAAGCTAAACGATGCTCAAGCACCAAAATAATA, SEQ ID NO. 8), B 2 (TTTTGGTGCTGAGCATCGTTTAGCTTTTTYIAGCTTCTGCTAAAATTTCTTT GCTCACTGAAGGATCGTCTTTAAGGCTTTGGATGAAGCC, SEQ ID NO. 9) and cloned into the BaeI-cleaved derivatives of pVSII described above. The resultant expression plasmids were designated pVS-HI-Cd, pVS-LL-Cd, pVS-PB10-Cd, pVS-PB40-Cd and pVS-PB80-Cd. Shuttle vectors containing these modified fiber genes were constructed by replacing the AgeIMfeI-fragment of the shuttle vector pKan_{ρ}HI by the AgeI-MfeI-fragments of pVS-HI-Cd, pVSLL-Cd, pVS-PB10-Cd, pVS-PB40-Cd and pVS-PB80-Cd.

### EXAMPLE 4

Expression of Ad5 Fiber Protein Modified with the C Domain of Staphylococcus aureus Protein A

The fiber-C domain genes were assembled in the mammalian expression plasmid pVS2 and the resultant recombinant vectors were then used to direct the expression of these genes in 293T/17 cells. These expression experiments were intended to demonstrate that the designed protein chimeras could be expressed at levels comparable with that of the wild type (wt) Ad5 fiber (Fbwt) and that they possess structural and functional properties required for both the incorporation of these proteins into Ad virions and for binding to Fc-containing proteins. 293T/17 cells were transfected with the pVS-derived expression vectors using the DOTAP liposomal transfection reagent (Roche, Mannheim, Germany) according to manufacturer's protocol. Seventy-two hours posttransfection, the cells were washed with PBS, harvested, and lysed in Cell Culture Lysis Reagent (Promega, Madison, Wis.) at 10⁶ cells/ml. Cell lysates were used for enzyme-linked immunosorbent analysis (ELISA) or immunoblotting.

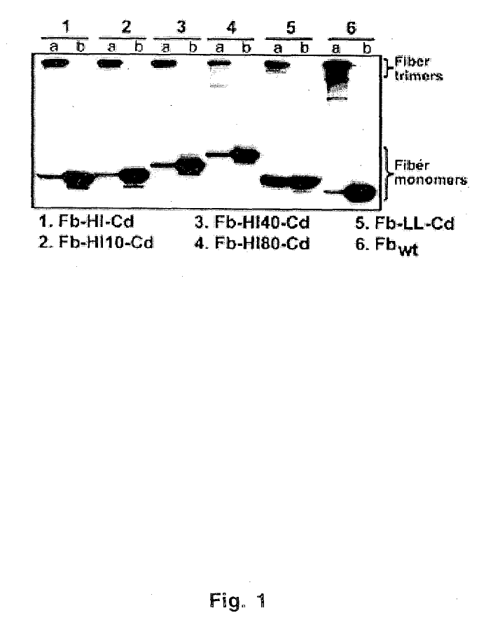

Immunoblotting of the lysates of pVS-transfected 293T/17 cells showed that the quantities of the fiber-C domain proteins were similar to the amount of the wt fiber expressed by the control plasmid (FIG. 1). A comparison of the mobilities of the chimeras in denatured and non-denatured samples clearly showed that all the newly designed proteins formed trimers upon self-association. Since trimerization of the fiber is a prerequisite of its association with the penton base protein, the results of this assay were indicative of the suitability of the fiber-C domain proteins for Ad capsid modification.

Next, Fc-binding capability of the C domain in the context of the fiber-C domain chimeras was examined. This was accomplished by an ELUSA which used the lysates of fiber-C domain-expressing 293T/17 cells for a binding assay employing the Fc-G28.5 protein as bait. The wells of 96-well Nunc Immuno-plates (Fisher Scientific, Pittsburgh, Pa.) were coated overnight at 4° C. with proteins diluted in 50 mM carbonate buffer (pH 8.6) at a concentration of 5 mg/ml. The unsaturated surface of the wells was then blocked for 1 h at room temperature by the addition of 200 ml of blocking buffer (Tris-buffered saline, TBS, with 0.05% Tween 20 and 0.5% casein) to each well. The blocking buffer was replaced with 100 ml of cell lysates or Ad preparations diluted in binding buffer (TBS with 0.05% Tween 20 and 0.05% casein). The plates were incubated at room temperature for 1 h and then washed four times with washing buffer (TBS with 0.05% Tween 20). Bound fiber proteins or Ad particles were detected by incubation for 1 h at room temperature with 4D2 mAb or anti-Ad2 polyclonal antibodies, respectively. The wells were washed four times with washing buffer and then either goat anti-mouse immunoglobulin G or goat anti-rabbit immunoglobulin antibodies conjugated with horseradish peroxidase (HRP) (Dako Corporation, Carpinteria, Calif.) were added and the incubation was continued for 1 h. The color was developed with Sigma FAST o-phenylenediamine dihydrochloride tablet kit (Sigma, St Louis, Mo.) as recommended by the manufacturer. Color intensity was measured at 490 nm with an EL800 plate reader (Bio-Tek Instruments, Winooski, Vt.).

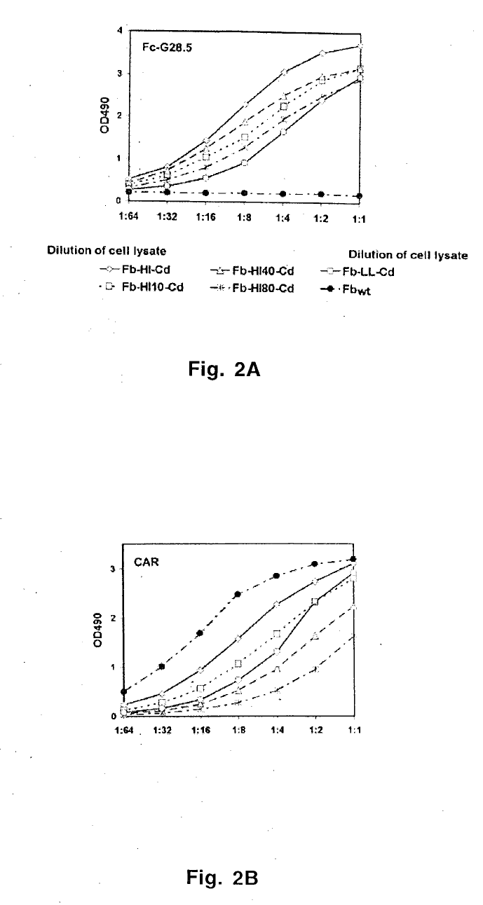

Results shown in FIG. 2A demonstrated that each of the fiber-C domain chimeras bound to the Fc domain, whereas the wild-type fiber did not bind to Fc-G28.5 even at the highest concentration used. In. addition, the interaction of the fiber-C domain proteins with CAR was examined. An ELISA employing a soluble form of CAR protein, sCAR, as the target showed that although the receptor-binding site within the modified fibers was affected by incorporation of C domain (FIG. 2B), all modified fibers largely retained the ability to bind CAR. Therefore, taken together, these experiments made it clear that despite very substantial modifications of the fiber structure, all five fiber-C domain proteins possess key functional properties that are essential for the realization of this Ad targeting scheme.

### EXAMPLE 5

Adenoviruses Containing Fiber Protein Modified with the C Domain of Staphylococcus aureus Protein A

Recombinant Ad genomes incorporating the modified fiber genes were derived by homologous DNA recombination in Escherichia coli BJ5183 with SwaI-linearized plasmid pVL3200 essentially as described previously (Chartier et al., 1996). pVL3200 is a derivative of pTG3602 (Chartier et al., 1996), which contains an Ad5 genome deleted for the E1, E3 and the fiber gene. In place of the deleted E1 it contains a cytomegalovirus immediate early promoter-driven expression cassette comprising the firefly luciferase gene and the green fluorescent protein gene linked with an internal ribosome entry site (IRES). The designations of the pVL3200-derived Ad vectors contain the abbreviation “DR”, such as Ad5.DR-LL-Cd, to reflect the presence of a double reporter (luciferase and GFP) in their genomes.

All Ad vectors were generated by transfection of 293 cells with PacI-digested Ad rescue vectors as described previously (Krasnykh et al., 1998). The viruses were propagated in 293 cells and purified by equilibrium centrifugation in CsCl gradients according to standard protocol (Graham and Prevec, 1995). Protein concentrations in viral preparations were determined by using the Dc protein assay (Bio-Rad, Hercules, Calif.) with purified bovine serum albumin (BSA) as a standard. Virus titers were calculated by using the formula: 1 mg of protein=4×10⁹ viral particles (vp).

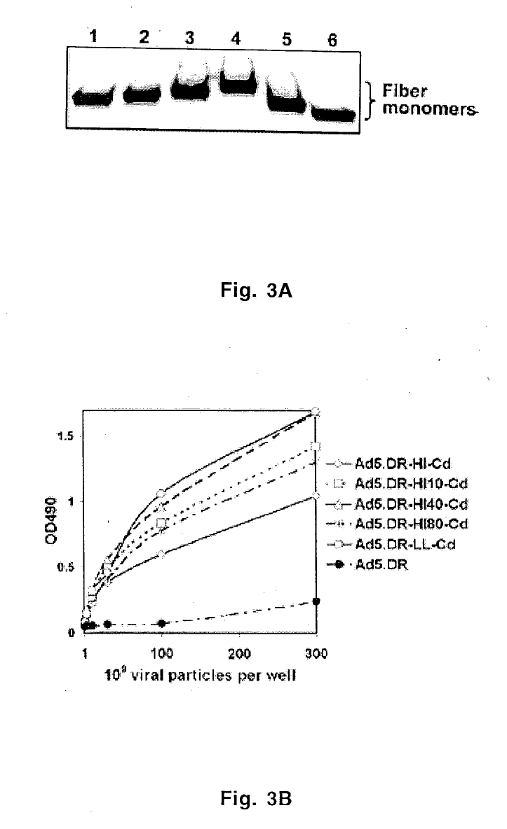

The dynamics of the infection by these vectors did not differ from those seen for a control Ad vector, Ad5.DR, incorporating wt fibers. As shown in Table 1, the titers of all six viruses were very similar. Also, as would have been predicted by the trimerization pattern of the transiently expressed fiber-C domain proteins, an immunoblot analysis of purified viruses showed efficient incorporation of these fiber chimeras into Ad capsids (FIG. 3A). Taken together, these observations suggested that the modifications of the fiber with C domain did not have any deleterious effect on the assembly of the virions.

TABLE 1
Yields of Ad.DR vectors in 293 cells.

| Virus | Particles per 10₈ cells |
|---|---|
| Ad5.DR | 1.1 × 10¹² |
| Ad5.DR-HI-Cd | 7.5 × 10¹¹ |
| Ad5.DR-HI10-Cd | 6.4 × 10¹¹ |
| Ad5.DR-HI40-Cd | 9.3 × 10¹¹ |
| Ad5.DR-HI80-Cd | 7.6 × 10¹¹ |
| Ad5.DR-LL-Cd | 8.5 × 10¹¹ |

### EXAMPLE 6

Construction of Fc-Single Chain Antibody Fusion Protein as Targeting Ligand

Having completed the modification of the Ad vectors, a complementary ligand molecule that would be capable of targeting the virus via association with its altered capsid was designed. To this end, the Fc domain of human Ig was employed as a fusion partner for a targeting single chain antibody (scFv) to generate a bifunctional “anchor-ligand” molecule. The role of the Fc domain in the present targeting scheme is two-fold. First, it is used to facilitate the expression and secretion of the targeting ligand; second, it also serves as an anchor that allows the ligand to associate with the C domain-modified Ad capsids.

The sequence encoding a fusion protein designated Fc-G28.5 comprising the secretory leader sequence, anti-CD40 single chain antibody (scFv) G28.5 (Pereboev et al., 2002) tagged with the Fc domain of human immunoglobulin and six-histidine sequence (6His) was assembled within the expression cassette of the AdApt shuttle vector (Crucell, Leiden, Netherlands). The Fc-G28.5-encoding gene was placed under transcriptional control of CMV5 promoter. The genome of Ad5.Fc-G28.5 containing this cassette in place of the deleted E1 region was then generated by homologous DNA recombination with the ClaI-linearized pTG3602 rescue vector.

To express Fc-G28.5, 6×10⁹ 293 cells were infected with Ad5.Fc-G28.5 at MOI of 100 vp/cell. The medium from the infected cells was collected at 72 h post infection and loaded onto a HiTrap rProtein A FF 5ml column (Amersham Biosciences, Piscataway, N.J.) equilibrated with phosphate-buffered saline (PBS). After washing the column with five column volumes of PBS, bound proteins were eluted with 0.1M Na-citrate, pH 3.4. To preserve the activity of the scFv, one milliliter fractions were collected into tubes with 200 ml of 1.5M Tris-HCl, pH 8.8. The collected protein was dialyzed against PBS and loaded onto a 1 ml HiTrap 6xHis FF column (Amersham). After washing the column with PBS, the protein was eluted with a linear gradient of imidazole (20 to 500 mM) in PBS. The protein was collected and dialyzed against PBS. The final protein concentration was determined using the Dc protein assay (Bio-Rad) with BSA as a standard.

A total of 6.8 mg of the fusion was purified in this way upon infection of 6×10⁹ 293 cells. Analytical gel filtration chromatography of Fc-G28.5 showed that it was present in the sample in a form of a dimer, which is typical of Fc-containing proteins. Electrophoresis of the resultant preparation showed that the Fc-G28.5 ligand was more than 95% pure (data not shown) and thus suitable for subsequent vector targeting experiments.

To confirm that both components of the newly designed gene delivery system, the viral vector and the targeting ligand, were able to associate with each other, an ELISA in which Fc-G28.5 used as bait was probed with purified Ad particles. As expected, this assay showed strong binding of each of the C domain-modified vectors to the ligand, while virtually no binding was observed with the control Ad lacking C domain in the capsid (FIG. 3B). Thus, these findings proved the feasibility of the formation of targeting vector complexes and therefore rationalized subsequent cell transduction studies.

In addition to the Fc-G28.5 protein, other targeting ligands can be constructed. The design, expression and purification of the recombinant protein comprising the extracellular domain of human CAR has been reported by Dmitriev et al. (Dmitriev et al., 2000). The expression of the 6His-tagged knob domain of Ad5 fiber in E. coli and its purification by immobilized ion metal affinity chromatography have been described previously (Krasnykh et al., 1996). All chromatographic separations were performed utilizing the ÄKTApurifier system on prepacked columns from Amersham Pharmacia Biotech Inc. (Piscataway, N.J.).

Recombinant protein Fc-CD40L, which consists of a genetic fusion of the DNA encoding the human tumor necrosis factor (TNF)-like domain of human CD40 Ligand sequence at its amino terminus to the hinge region of the Fc domain of human IgGg1, was expressed in murine NS/0 cells and purified as previously described (Lo et al., 1998).

### EXAMPLE 7

Preliminary Assessment of Gene Transfer Properties of Ad::ligand Targeting Complexes

A comparison of the gene delivery characteristics of the Ad::Fc-G28.5 complexes was done by means of a transduction experiment employing 293.CD40 cells as the target. Since all the Ad vectors used in these studies contained fibers with functional CAR-binding sites, CAR on the surface of the target cells were blocked with knob protein (Krasnykh et al., 1996) in order to discriminate between CAR-mediated cell entry versus that which was expected to result from the attachment of the targeting complexes to CD40. Prior to infection with the modified Ad vectors, the cells were preincubated with either medium alone, medium containing recombinant Ad5 fiber knob protein, or medium containing the knob and Fc-G28.5 ligand. Ad vectors incorporating wt fibers, and parental 293 cells that do not express any detectable CD40 were employed as negative controls.

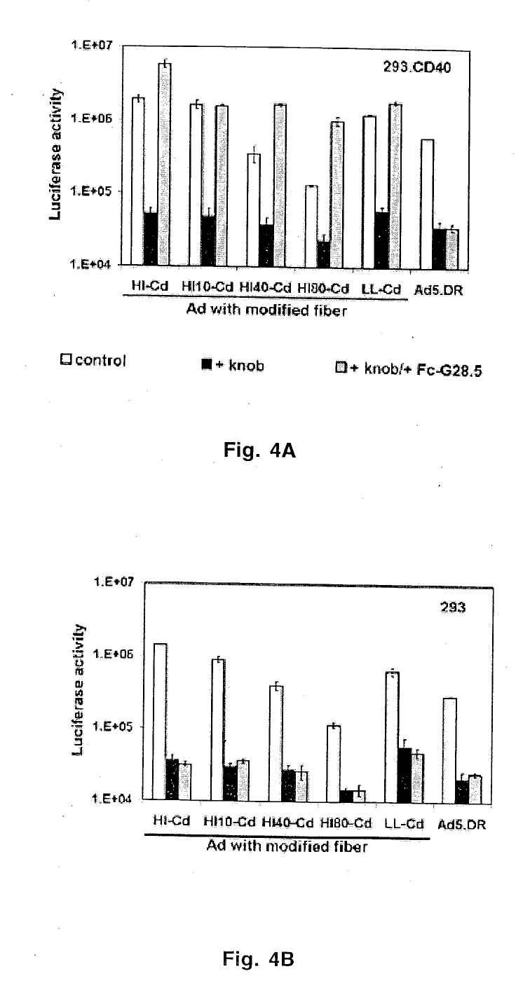

This experiment showed that all C domain-modified Ad were able to employ the Fc-G28.5 ligand for CD40-mediated infection, with no significant variations between the vectors (FIG. 4). These data obviated the need to continue the work with all five modified vectors. Therefore, Ad5.DR-HI10-Cd, Ad5.DR-HI40-Cd, and Ad5.DR-LL-Cd were chosen for the following experiments, as these constructs represented two different Ad fiber modification approaches: the redesign of the HI loop and the carboxy terminus of the protein.

### EXAMPLE 8

Preparation and Characterization of Preformed Ad::ligand Complexes

Complexes of Ad with Fc-containing targeting ligands were generated during purification of viruses from infected 293 cells. Briefly, 293 cells were infected with adenoviruses at a multiplicity of infection (MOI) of 300 vp/cell. Cells were harvested at 55 h post-infection and resuspended in 2% FBS/DMEM. Viruses were released from the cells by three freeze-thaw cycles, and the cell debris was removed by centrifugation. The supernatant was layered onto a preformed step gradient of CsCl and centrifuged at 25,000 rpm for 3 h at 4° C.. Banded viruses were collected, mixed with Fc-G28.5 or Fc-CD40L proteins at a concentration of 30 mg/ml and incubated for 30 min at room temperature. All the C domain anchoring sites within the virions are expected to be occupied by the targeting ligands under high ligand-to-virus ration. Vector complexes were purified from unbound proteins by equilibrium centrifugation in CsCl gradients, dialyzed (10 mM Tris-HCl, pH8.0, 50 mM NaCl, 2 mM MgCl₂, 10% glycerol) and stored at −80° C. until use.

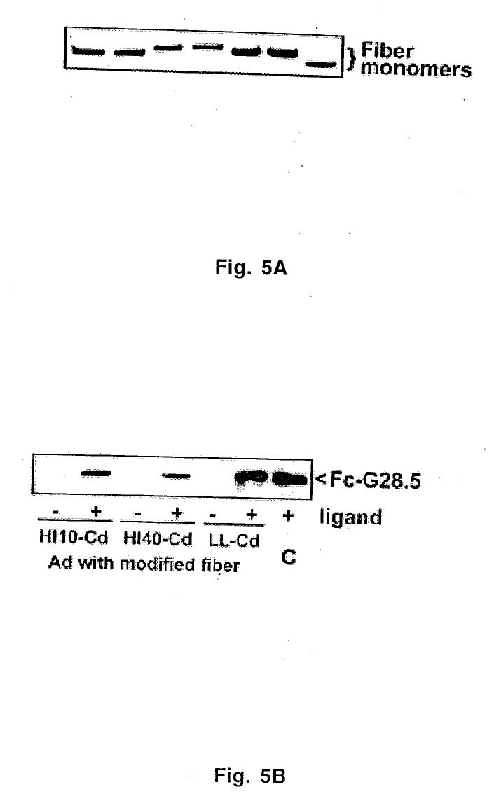

Each of the three viruses, Ad5.DR-HI10-Cd, Ad5.DR-HI40-Cd, and Ad5.DR-LL-Cd, was mixed and incubated with the targeting Fc-scFv ligand as described above. The efficiency of association of the ligand with each of the viruses was examined in an immunoblot assay using a Penta-His mAb that binds to the 6His tag present in the ligand molecule. This analysis showed that Fc-G28.5 protein bound most efficiently to Ad5.DR-LL-Cd, while the amount of the ligand found in preparation of Ad5.DRHI10-Cd and Ad5.DR-HI40-Cd was lower (FIG. 5).

### EXAMPLE9

Transduction Properties of the Preformed Ad::Ligand Complexes on Established Cell Lines

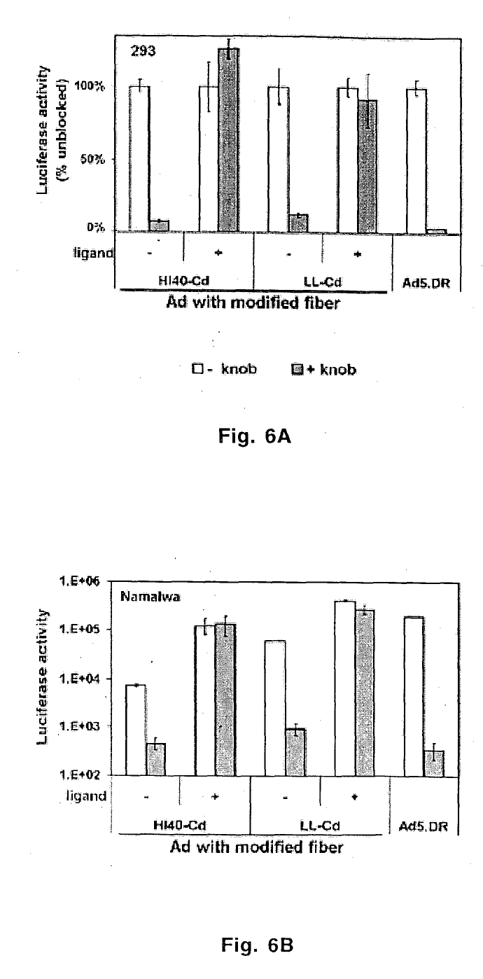

The receptor specificity of the resultant vector complexes was assessed by employing them to infect two different cell targets. First, these complexes were used to transduce 293 cells, which are CAR-positive but do not express any detectable CD40. The main purpose of this experiment was to test whether the 5 association of Ad vectors with the ligand affected the viruses' ability to bind CAR. Ad5 fiber knob protein was added to duplicate samples to block CAR receptors present of the cells. Predictably, when used without a ligand, each of the viruses was capable of using CAR for cell entry, as evidenced by efficient inhibition by the knob protein. In contrast, the infectivity of Ad::Fc-G28.5 vector complexes was not affected by the presence of the knob (FIG. 6A).

These vectors were then employed for infection of Namalwa human lymphoblastoid cells, which are CAR-positive and naturally express CD40. As seen in FIG. 6B, the vector complexes clearly outperformed the relevant untargeted Ad, with the difference in the infection efficiencies being in the range of an order of magnitude for each vector. Importantly, this augmentation of infectivity was entirely due to targeting of the vectors to CD40, as the addition of the fiber knob protein had no effect on gene transfer. Of special note, Ad5.DR-HI10-Cd demonstrated an infection profile which was very similar to that of Ad5.DR-HI40-Cd (not shown).

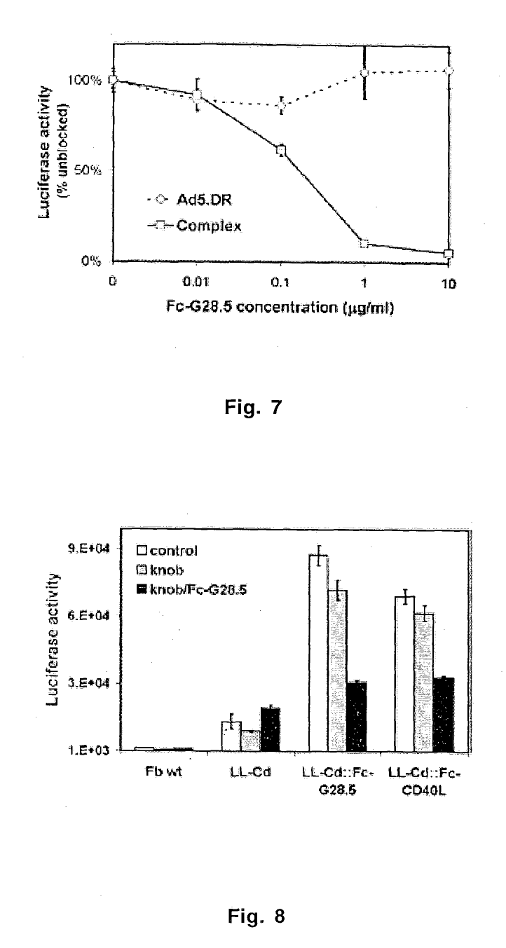

The CD40-dependence of the infection by the targeted complexes was further confirmed by transducing Namalwa cells with Ad5.DR-LL-Cd::Fc-G28.5 in the presence of various concentrations of free ligand. This resulted in a Fc-G28.5 concentration-dependent inhibition of transduction, which unambiguously demonstrated the direct involvement of CD40 in the cell entry pathway used by the ligand-containing vector complex (FIG. 7). As expected, the infectivity of the Ad5.DR vector, which contains wild type fibers and is thus unable to associate with Fc-G28.5, was not affected by the addition of the free ligand.

### EXAMPLE 10

In Vitro Transduction of Primary Human Dendritic Cells with the CD40-Targeted Vectors

An additional test of the cell transduction ability of the Ad5.DR-LL-Cd::Fc-G28.5 vector was done using human dendritic cells (DCs) as targets. These DCs were derived from CD14-positive monocytes isolated from human peripheral blood. For the purpose of comparison, a similarly prepared vector complex containing the CD40-binding domain of human CD40 Ligand, CD40L, fused with Fc was also employed. This experiment demonstrated that, when complexed with either of the two targeting ligands, the C domain-modified vector was able to deliver the reporter gene to dendritic cells 28- to 35-fold more efficiently than the control unmodified vector, Ad5.DR (FIG. 8).

In line with previous reports of poor expression of CAR and elevated levels of CD40 in dendritic cells, the use of the Ad5 fiber knob and scFv_{G28.5} as inhibitors of infection revealed that the CD40-mediated component of overall gene transfer by the targeted vectors was higher than that involving CAR, which was observed for untargeted Ad. On another note, the scFv_{G28.5} constituent of the targeting protein was more efficient in directing the vector complex to dendritic cells than was the natural ligand of CD40, CD40L, thus further supporting the choice of scFvs as targeting moieties for Ad.

### EXAMPLE 11

Construction of Targeted Adenoviral Vector for Selective Expression of Tumor-Specific Antigen in Dendritic Cells

The following example describes the construction of targeted adenoviral vector for selective expression of tumor-specific antigen in dendritic cells. The cloning procedure involves the following steps:

generating an Ad shuttle vector containing an expression cassette incorporating genes encoding a tumor-specific antigen and a targeting ligand;

incorporating the dual expression cassettes into a fiber gene-deleted, green fluorescent protein-expressing Ad genome;

cloning of mammalian expression plasmids incorporating genes encoding for Ad fibers modified with the C-domain of S. aureus protein A (CdpA);

transient expression of the fiber-CdpA proteins in 293T cells for structural integrity assessment;

transferring the fiber-CdpA-encoding genes into an Ad fiber shuttle vector;

transferring the fiber-CdpA-encoding genes from the Ad fiber shuttle vectors into the fiber gene-deleted Ad genome expressing the tumor-specific antigen and the targeting ligand; and

rescue and amplification of the viruses of interest.

Adenoviral shuttle vector containing an expression cassette incorporating genes encoding a targeting ligand and a tumor-specific antigen is constructed as follows. The vector is designed using the Ad shuttle plasmid which contains an expression cassette driven by the strong cytomegalovirus promoter. First, the expression cassette within the plasmid is duplicated and multiple cloning sites within one of the two cassettes is replaced with a synthetic DNA sequence containing a set of alternative cloning sites. The plasmid containing this double cassette will allow the cloning of transgenes into either of the two polylinker sequences. DNA sequence encoding a tumor-specific antigen, such as the cDNA of prostate-specific membrane antigen, is cloned into one of the cassettes. Subsequently, sequence encoding fusion proteins comprising either the soluble form of CD40L (sCD40L) or anti-CD40 scFv G28.5 tagged with the Fc domain of human immunoglobulin is cloned into the other cassette. This targeting ligand is designed to target Ad vectors incorporating within their capsids C-domain of S. aureus protein A. All targeting ligand-encoding sequences described here are designed by the “sticky end” PCR technique.

The dual expression cassette is then incorporated into a fiber gene-deleted, green fluorescent protein-expressing Ad genome. First, the E3 region of an Ad5 genome contained in the Ad rescue vector pVK is replaced with an expression cassette containing the green fluorescent protein (GFP) gene. This is followed by incorporating the dual expression cassettes constructed above in place of the E1 regions of the Ad genome contained in the resultant rescue plasmid. Transfer of all transgenes into the Ad genome is done by the method of homologous DNA recombination in bacteria originally described by Chartier et al. (1996).

To construct mammalian expression plasmid incorporating gene encoding Ad fiber modified with the C-domain of S. aureus protein A (CdpA), CdpA can be genetically fused with either the carboxy terminus of the previously described Ad5 fiber:T4 fibritin protein chimera (Krasnykh et al., 2001), or the HI loop of the Ad5 fiber knob domain. Sequence encoding the C domain is cloned into the BaeI-cleaved mammalian expression vectors pVS.F_{cBaeI} or pVS.F_{FBae}I, which contain the genes for the fiber and fiber:fibritin, respectively. As a result of this cloning step, the open reading frames of each of the two carrier proteins will be fused with that of the C domain.

The fiber-fibritin chimera is employed as an alternative strategy to generate the fiber-C domain chimeric gene. The fiber-fibritin protein was designed so that the structure of the domain providing for trimerization of the chimera (fibritin) is not affected by incorporation of heterologous peptides/polypeptides within the protein, thereby dramatically increasing the odds of obtaining stable derivatives of this “backbone” molecule. This strategy of fiber replacement has been described in a recent paper (Krasnykh et al., 2001).

The expression plasmids of the pVS series described above can be used to direct production of the C domain-modified fibers in mammalian cells. For this 293T cells are transfected with each of the pVS vectors and the expression of the fiber-C domain proteins is assessed 48 hrs later by lysing the cells and analyzing their lysates by Western blot with anti-fiber tail mAb 4D2. As the trimeric structure of Ad fiber is a prerequisite for its successful incorporation into an Ad virion, this assay will allow us to identify those fiber-C domain species that can be employed for the Ad targeting disclosed herein.

The expression plasmids of the pVS series are designed to be “compatible” with the fiber shuttle vectors of the pKan series to insert modified fiber genes into Ad genomes. Those fiber-C domain genes whose products have successfully passed the trimerization test are cloned into the pKan vectors in a simple subcloning step utilizing the same pair of restriction enzymes (MfeI and AgeI) for all constructs to be made.

The genes encoding the newly designed fiber-C domain proteins are then incorporated into the Ad rescue vectors constructed above by homologous DNA recombination in bacteria. The fiber-C domain genes are incorporated into Ad genomes containing the genes for Fc-ligands, whereas zipper-fiber genes are inserted into the genomes incorporating zipper-Fc-ligand genes. Consequently, the design of Ad genomes of interest is completed and the viruses of interest are rescued and amplified in 293 cells.

### EXAMPLE 12

Induction of Dendritic Cells Maturation Upon CD40-Mediated Infection

The following example examines the effects of vector targeting to CD40 on the phenotype of dendritic cells. It is expected that not only can CD40-targeted vectors deliver antigen-expressing genes to dendritic cells in a more efficient manner, but also that they are able to trigger maturation and activation of dendritic cells and thus launch the generation of an immune response. In this regard, it is known that activated dendritic cells have a characteristic phenotype, which can be shown by flow cytometry and also confirmed functionally by examination of the cytokines they secrete and the cytokines they induce T cells to secrete. In addition, activation of naive CD4⁺ T cells is a hallmark of dendritic cell function. These functions can be examined by various immunologic assays described below.

Day 5 dendritic cells (DCs) are transduced with CD40-targeted Ad vectors or control Ad lacking targeting capacity. Twenty-four hours later aliquots of dendritic cells are subjected to fluorescence-activated cell sorting (FACS) for analysis of CD40, CD54, CD80, CD86 (T cell co-stimulatory markers), CD83 (DC maturation marker), CCR7 (lymph node homing marker) and CCR6 (immature DC marker ) expression. It is expected that targeted Ad vectors will induce DC maturation/activation significantly better than control Ad, as will be evidenced by increased expression of CD40, CD54, CD80, CD83 and CD86. CCR6 expression is expected to be downregulated, while the mature DC marker CCR7 is expected to be expressed at an elevated level. CCR7 is associated with lymph node homing, and thus increased CCR7 expression can improve in vivo immunogenicity of transduced DCs.

Dendritic cell function can be assessed by two independent means: i) analysis of secreted DC products and ii) analysis of effects on T cell function. Myeloid DCs secrete IL-12 upon activation to induce a strong Th1 polarized immune response dominated by T cell interferon-g. IL-10 is also induced and this can reduce induced interferon-g. Day 5 dendritic cells are transduced with adenovirus as above and IL-12 and IL-10 are measured in the supernatant 24 hours later by ELISA (R&D Systems). Controls include non-targeted vector and “no treatment” as negative controls. Lipopolysaccharide from E. coli LPS is used at 100 ng/ml as a positive control. CD40-targeted Ad vectors are expected to induce DC maturation/activation significantly better than those not targeted to CD40, as will be evidenced by an increased capacity of DCs to secrete IL-12. IL-10 may also be induced, but not at higher levels than in control samples.

T cells activated by myeloid dendritic cells secrete significant amounts of interferon-g and IL-2, with little IL-4 and no IL-10. T cells are activated by incubation with Ad-transduced day 5 dendritic cells 24 hours post transduction. Induced cytokines can be assessed at single cell level by in situ cytokine detection assay as previously described (Zou et al., 2000, 2001), and confirmed by ELISA of supernatants. T cell activation are confirmed by proliferation in an allogeneic mixed lymphocyte reaction (MLR). Here, naïve CD⁴⁺ CD62L⁺ CD45RO⁻ CD4⁺ T cells are isolated using beads (Miltenyi) as described (Zou et al., 2000, 2001), and MTT dye uptake and total cell numbers are measured 3 days later.

Tumor-specific CTLs are thought to be pivotal effectors in specific immunity. CTL-inducing capacity of dendritic cells transduced with targeted Ad vectors can be examined by a generic approach and a tumor-specific approach. For the generic approach, interferon-g⁺ CD8⁺ T cells, which are accepted surrogates of CD8⁺ CTLs, can be detected by flow cytometry as described (Zou et al., 2000). Allogeneic CD8⁺ T cells are incubated with Ad-transduced dendritic cells and interferon-g⁺ CD8⁺ T cells can be detected by flow cytometry 3 days later.

Prostate-specific membrane antigen (PSMA)-specific immunity can be examined using peripheral blood CD3⁺ total T cells induced to proliferate with 2 HLA A2-restricted peptides. Tetramers for these peptides can be synthesized as previously described (Altman et al., 1996). Influenza matrix₅₈₋₆₆ peptide (which binds to HLA A2) is used as a control. Tetramer complexes can be combined with PE, or allophycocyanin (APC)-labeled streptavidin, and tetramer+cells are analyzed by FACS. These studies can be confirmed with cytotoxicity assays using [⁵¹Cr]-labeled T2 cell lines (ATCC) pulsed with or without the HLA-A2-restricited PSMA peptides as targets in standard [⁵¹Cr] release assay. Negative controls include T2 cells pulsed with influenza matrix₅₈₋₆₆ peptide and T2 cells with no peptide. Consistent with the mature/activated phenotype of Ad-transduced dendritic cells, it is expected that they will activate a higher level of T cell proliferation and induce significant levels of interferon-g and IL-2 production by T cells. As CD40 ligation enhances CTL activity, it is also expected that dendritic cells activated by the CD40-targeted Ad will exhibit better CTL activity compared to dendritic cells transduced with non-targeted Ad.

### EXAMPLE 13

The Ability of CD40-Targeted Ad Vectors to Induce Maturation and Migration of Human Dendritic Cells

Dendritic cells naturally present in human skin mimic the anticipated use of DC-targeted Ad vectors for immunization via intradermal injection. The goal of following studies is to show that targeting of Ad vectors to dendritic cells via the CD40-pathway allows the vectors to find and selectively transduce their cell targets (DCs) in a complex context of a real human tissue.

Skin explants cultured with the epidermal side up on filter-covered grids over a period of 24 hours are injected with CD40-targeted Ad vectors or plain medium. The explants are placed in culture medium (floating with the epidermal side up) in a 48-well culture plate and further incubated before migrating dendritic cells are harvested. Subsequent studies including cytometry, immunohistochemistry and MLR performed according to protocols well known in the art.

The following references were cited herein:

Altman et al., Phenotypic analysis of antigen-specific T lymphocytes. Science 274:94-6 (1996).

Belousova et al., Modulation of adenovirus vector tropism via incorporation of polypeptide ligands into the fiber protein. J Virol. 76:8621-31 (2002).

Chartier et al., Efficient generation of recombinant adenovirus vectors by homologous recombination in Escherichia coli. J Virol. 70:4805-10 (1996).

Dmitriev et al., Ectodomain of coxsackievirus and adenovirus receptor genetically fused to epidermal growth factor mediates adenovirus targeting to epidermal growth factor receptor-positive cells. J Virol. 74:6875-84 (2000).

Graham and Prevec, Methods for construction of adenovirus vectors. Mol. Biotechnol. 3:207-20 (1995).

Heiser et al., Autologous dendritic cells transfected with prostate-specific antigen RNA stimulate CTL responses against metastatic prostate tumors. J Clin. Invest. 109:409-17 (2002).

Hong and Engler, Domains required for assembly of adenovirus type 2 fiber trimers. J Virol. 70:7071-8 (1996).

Krasnykh et al., Genetic targeting of adenovirus vector via replacement of the fiber protein with the phage T4 fibritin. J. Virol. 4176-4183 (2001).

Krasnykh et al., Genetic targeting of adenoviral vectors. Mol. Ther. 1:391-405 (2000).

Krasnykh et al., Characterization of an adenovirus vector containing a heterologous peptide epitope in the HI loop of the fiber knob. J Virol. 72:1844-52 (1998).

Krasnykh et al., Generation of recombinant adenovirus vectors with modified fibers for altering viral tropism. J Virol. 70:6839-46 (1996).

Krasnykh and Douglas, Targeted adenoviral vectors I: Transductional targeting. In Curiel and Douglas ed., Adenoviral Vectors for Gene Therapy. Academic Press, San Diego (2002).

Lo et al., High level expression and secretion of Fc-X fusion proteins in mammalian cells. Protein Eng. 11:495-500 (1998).

Lodge et al., Expression and purification of prostate-specific membrane antigen in the baculovirus expression system and recognition by prostate-specific membrane antigen-specific T cells. J Immunother. 22:346-55 (1999).

Meidenbauer et al., Generation of PSA-reactive effector cells after vaccination with a PSA-based vaccine in patients with prostate cancer. Prostate 43:88-100 (2000).

Pereboev et al., Coxsackievirus-adenovirus receptor genetically fused to anti-human CD40 scFv enhances adenoviral transduction of dendritic cells. Gene Ther. 9:1189-93 (2002).

Salgaller et al., Dendritic cell-based immunotherapy of prostate cancer. Crit. Rev. Immunol. 18:109-19 (1998).

Tasch et al., A unique folate hydrolase, prostate-specific membrane antigen (PSMA): a target for immunotherapy? Crit. Rev.

Immunol. 21:249-61 (2001).

Tillman et al., Adenoviral vectors targeted to CD40 enhance the efficacy of dendritic cell-based vaccination against human papillomavirus 16-induced tumor cells in a murine model. Cancer Res. 60:5456-63 (2000).

Tillman et al., Maturation of dendritic cells accompanies high-efficiency gene transfer by a CD40-targeted adenoviral vector. J Immunol. 162:6378-83 (1999).

Tjoa and Murphy, Progress in active specific immunotherapy of prostate cancer. Semin. Surg. Oncol. 18:80-7 (2000).

Von Seggern et al., J Virol. 74:354-62 (2000).

Zou et al., J Immunol. 165:4388-96 (2000).

Zou et al., Nat Med. 7:1339-46 (2001).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A targeted recombinant adenovirus vector, comprising:
(i) a gene encoding a heterologous protein;
(ii) a modified fiber protein comprising an immunoglobulin-binding domain; and
(iii) a gene encoding a fusion protein comprising a targeting ligand and an immunoglobulin Fc domain, wherein binding of said immunoglobulin-binding domain to said Fc domain connects said targeting ligand to said modified fiber protein, thereby targeting said adenovirus vector to a cell that expresses a cell surface molecule that binds to said targeting ligand.

2. The targeted adenovirus vector of claim 1, wherein said immunoglobulin-binding domain is the Fc-binding domain of Staphylococcus aureus Protein A.

3. The targeted adenovirus vector of claim 1, wherein said immunoglobulin-binding domain is inserted at the HI loop or the carboxy terminal of said fiber protein.

4. The targeted adenovirus vector of claim 3, wherein said immunoglobulin-binding domain inserted at the HI loop is flanked by flexible linkers.

5. The targeted adenovirus vector of claim 1, wherein said fiber protein is a fiber-fibritin chimera, and said immunoglobulin-binding domain is inserted at the carboxy terminal of said fiber-fibritin chimera.

6. The targeted adenovirus vector of claim 1, wherein said targeting ligand is selected from the group consisting of CD40 ligand and a single chain fragment (scFv) of anti-human CD40 antibody.

7. The targeted adenovirus vector of claim 1, wherein said heterologous protein is a tumor associated antigen.

8. The targeted adenovirus vector of claim 1, wherein said tumor associated antigen is prostate-specific membrane antigen.

9. A CD40-targeted recombinant adenovirus vector, comprising:
(i) a gene encoding a heterologous protein;
(ii) a modified fiber protein comprising an immunoglobulin-binding domain; and
(iii) a gene encoding a fusion protein comprising an immunoglobulin Fc domain and a targeting ligand selected from the group consisting of CD40 ligand and a single chain fragment (scFv) of anti-human CD40 antibody, wherein binding of said immunoglobulin-binding domain to said Fc domain connects said targeting ligand to said modified fiber protein, thereby targeting said adenovirus vector to a CD40⁺ cell.

10. The targeted adenovirus vector of claim 9, wherein said immunoglobulin-binding domain is inserted at the HI loop or the carboxy terminal of said fiber protein.

11. The targeted adenovirus vector of claim 10, wherein said immunoglobulin-binding domain inserted at the HI loop is flanked by flexible linkers.

12. The targeted adenovirus vector of claim 9, wherein said immunoglobulin-binding domain is the Fc-binding domain of Staphylococcus aureus Protein A.

13. The targeted adenovirus vector of claim 9, wherein said fiber protein is a fiber-fibritin chimera, and said immunoglobulin-binding domain is inserted at the carboxy terminal of said fiber-fibritin chimera.

14. The targeted adenovirus vector of claim 9, wherein said CD40⁺ cell is a dendritic cell.

15. The targeted adenovirus vector of claim 9, wherein said anti-human CD40 antibody is G28.5.

16. The targeted adenovirus vector of claim 9, wherein said heterologous protein is a tumor associated antigen.

17. The targeted adenovirus vector of claim 16, wherein said tumor associated antigen is prostate-specific membrane antigen.

18. The targeted adenovirus vector of claim 9, wherein said gene encoding said heterologous protein and said gene encoding said fusion protein are operably linked to a dendritic cell-specific promoter.

19. A method of gene transfer to CD40⁺ cells, comprising the step of:
contacting said cell with the targeted adenovirus vector of claim 9, wherein said targeted adenovirus vector mediates transfer of said gene encoding said heterologous protein to said cell.

20. The method of claim 19, wherein said CD40⁺ cells are dendritic cells.
